(19) United States
(12) Patent Application Publication
(10) Pub. No.: US 2005/0061696 A1
(43) Pub. Date: Mar. 24, 2005

(54) Surgical instrument organizer

(76) Inventor: Ronald Swank, Southampton, PA (US)

Correspondence Address: Mark S. Leonardo, Esq.;Brown Rudnick Berlack Israels LLP, One Financial Center, Boston, MA, 02111, US

(21) Appl. No.: 10/838,013

(22) Filed: May 3, 2004

Related U.S. Application Data
(60) Provisional application No. 60/467,826, filed on May 2, 2003.

(51) Int. Cl. A61L 15/00

(52) U.S. Cl. 206/363; 206/372; 206/438

(57) ABSTRACT

A slotted disposable foam instrument holder is provided for use in organizing surgical instruments wherein the surgical instruments are easily organized and available for efficient use during surgical operation. The inventive slotted disposable foam instrument holder provides the surgical technician with a uniform organizer that makes pre and post surgical counts accurate. The slotted nature of the organizer allows for the secure placement of surgical instruments preventing the toppling of the instruments during the surgical procedure and helps maintain the instruments in an organized and upright state.

### RELATED APPLICATIONS

This application claims priority to U.S. Provisional Application No. 60/467,826 filed on May 2, 2003, which is incorporated in its entirety by reference.

### FIELD OF THE INVENTION

The present disclosure is directed to an organizer for separating and securing surgical instruments before and during surgery and in preparation for sterilization. More specifically, the present disclosure relates to a polymeric foam organizer for separating, securing and maintaining surgical instruments in an upright position for easy access and accountability before and after a surgical procedure.

### BACKGROUND OF THE INVENTION

A surgical team utilizes a vast variety of surgical instruments during any surgical procedure. Surgical instruments are pre-selected, packaged, sterilized and delivered to the operating room. These sterilized packages are carefully opened and counted by surgical technicians and then arranged in the sterile field which consists of a draped stand known as a mayo table over the surgical site, and a longer larger back table for the specialized instruments. In the average procedure numerous ring handled instruments are positioned on the stand over the surgical incision and typically laid out on a rolled towel; the same way it has been done for about 100 years.

The rolled-up cotton towels, which may have their outside edges taped, support the instruments such that the instruments can be more easily counted, selected and handled by a doctor or nurse during a surgical procedure, or while the instruments are assembled prior to sterilization.

Recently, foam organizers have also been used to support and group surgical instruments before and during actual use of the instruments. Such organizers typically have an elongated base with fixed posts extending upwardly from ends of the base. These foam organizers support the instruments, with the instruments resting on the base and leaning against the posts, such that the instruments can be more easily counted, selected and handled by a doctor or technician during a surgical procedure, or while the instruments are assembled prior to sterilization. Additionally these foam organizers have also been fitted with a moveable post assembly that allows the technician to support surgical instruments by leaning the looped handle against the movable post.

Such rolled cotton towels or foam organizers, with the surgical instruments supported thereon, are usually laid out on a tray or a table. Unfortunately, however, before and during use of the surgical instruments, the instruments supported on the rolled towel or foam organizer can topple over from their upright positions and become mixed and disorganized. This tendency of surgical instruments to topple during use of these prior art organizer and towel, undesirably cause the instruments to be more difficult to count, select and handle prior to their use.

In addition, it is not uncommon for the cotton towels to produce lint, which can transmit microorganisms and result in contamination of the surgical instruments thereby affecting the integrity of the sterile surgical environment.

Therefore, there exists a need in the art of an organizer having a means for retaining surgical instruments in an organized and/or upright position on an instrument organizer. Further it is desirable to provide an instrument organizer where instruments supported thereon are less likely to topple over from an upright position prior to actual use or sterilization of the instruments and are easier to count, select and handle. Preferably, the slotted instrument organizer will be lint-free and made of materials that provide a cost effective disposable organizer.

### SUMMARY OF THE INVENTION

Accordingly, a slotted disposable foam instrument holder is provided for use in organizing surgical instruments wherein the surgical instruments are easily organized and available for efficient use during surgical operation. Additionally, the slotted disposable foam instrument holder, according to the invention, provides the surgical technician with a uniform organizer that makes pre and post surgical counts efficient. The slotted disposable foam allows for the secure placement of surgical instruments preventing the toppling of the instruments during the surgical procedure. The slotted nature of the organizer helps maintain the instruments in an organized and upright state.

According to one aspect of the present disclosure, the instrument organizer is made of lint-free foam polymer. According to another aspect of the present disclosure the instrument organizer is non-sterile and disposable. According to a further aspect of the present disclosure, the instrument organizer is provided with an adhesive strip or Velcro® strip on a bottom surface thereof for securing the organizer to a surface, such as a tray or table top for example.

Still other features and advantages of the presently disclosed surgical instrument organizer and method for sterilizing surgical instruments will become apparent upon reading the following detailed description in conjunction with the attached drawings.

### BRIEF DESCRIPTION OF THE DRAWINGS

FIG. 1 shows an exploded perspective view of an instrument organizer, including a slotted incision according to the present disclosure;

FIG. 2 shows a perspective view of the instrument organizer of FIG. 1 supporting surgical instruments in an upright position, with the slotted foam area providing support to the instruments; and

FIGS. 3A, 3B and 3C showing the inventive slotted organizer in use an operating room.

### DETAILED DESCRIPTION OF THE INVENTION

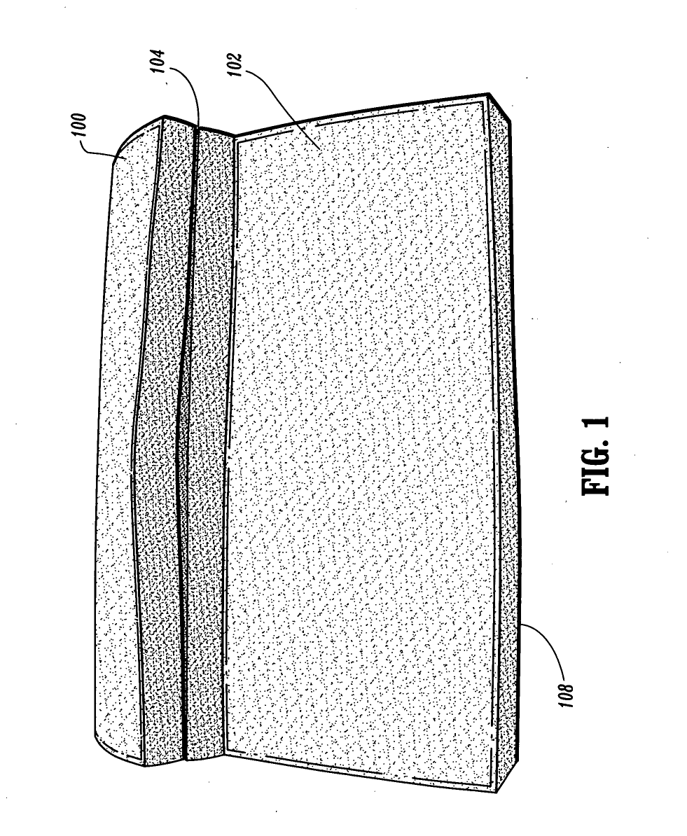

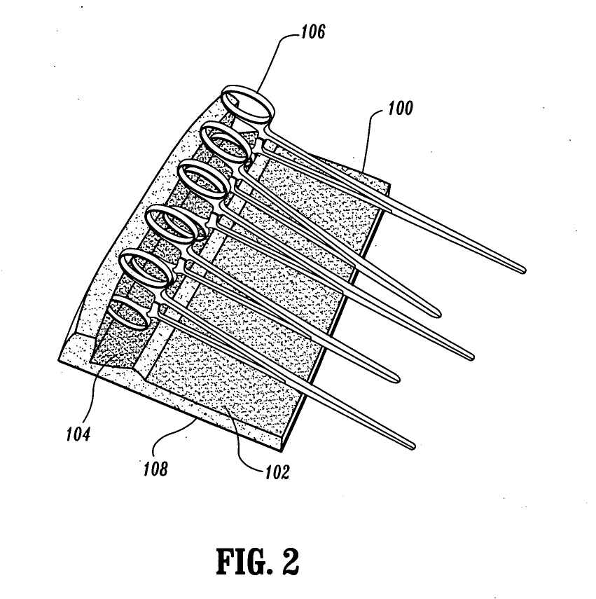

Referring to FIGS. 1 and 2, a slotted instrument organizer 100 according to the present disclosure is shown being used. According to the invention, a polymeric foam base 102 having a slotted incision 104 allows the polymeric foam base 102 to provide a means for holding ringed portions 106 of a surgical instrument. It is contemplated within the scope of the invention that the foam base 102 having the slotted incision 104 can secure instruments having non-ringed handles.

The slotted instrument organizer 100 provides the benefit of maintaining instruments supported by the organizer in an organized and upright manner to ensure that the instruments can be more easily organized, counted, selected and handled by a doctor or technician during a surgical procedure. Further the slotted instrument organizer 100, in one illustrative embodiment is fabricated from a lint free polymer. This lint free material enhances an operating team's assurance that the organized surgical instruments will be contamination free. Preferably, the slotted instrument organizer 100 is made of a non-woven, non-absorbent, lint-free material. The organizer 100 is preferably made of a lint-free foam polymer, such as ReticuCel® foam or the like. It is contemplated within the scope of the invention that the organizer 100 can be made of various polymeric materials known in the art that provide resilience and flexibility allowing for secure placement of surgical instruments. In a further illustrative embodiment the slotted instrument organizer 100 is formed of polymeric material having anti-microbial properties. In yet a further illustrative embodiment the polymeric material of the instrument organizer 100 is formed of a polymeric material that is heat resistant allowing for sterilization of organized surgical instruments. In addition, it is envisioned within the scope of the invention that the organizer can be provided in a sterile state or in a non-sterile state.

Referring in particular to FIG. 2, the organizer 100 can also be provided with a double-sided adhesive strip (not shown) on a bottom surface 108 of the base of the organizer 100. The adhesive strip allows the organizer 100 to be secured to a planar support surface such as a tray or table top for example. The adhesive tape allows the user to hold the organizer 100 in place and further prevent surgical instruments supported thereon from toppling over and becoming disorganized. The adhesive strip may further be provided in a sterile state. It is also contemplated within the scope of the invention that the organizer can be fitted with a Velcro® strip to attachment to a planar support surface having a receiving Velcro strip. It is further contemplated within the scope of the invention that the organizer 100 can have an adhesive backing upon its bottom surface 108 having a selected tack to prevent the organizer 100 from moving.

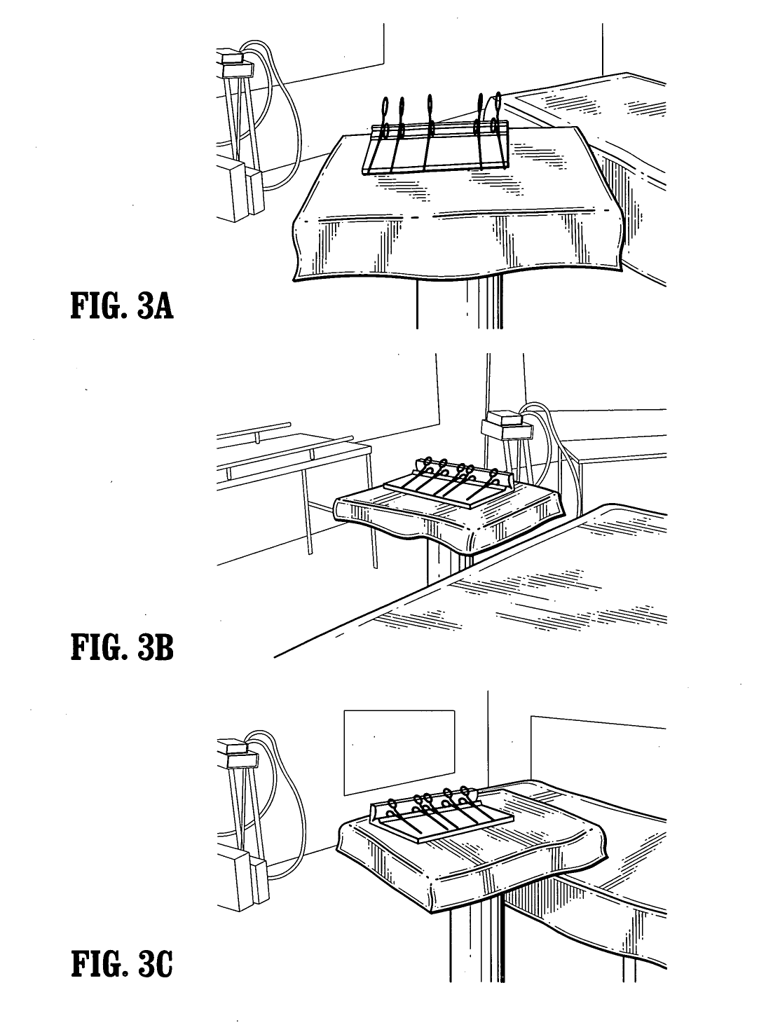

Turning to FIGS. 3A, 3B and 3C the slotted foam organizer is shown within the operating room environment. As shown in the operating room environment, the slotted organizers can be a variety of lengths that can be custom cut to organize various surgical instruments. It is contemplated within the scope of the invention that the polymeric foam forming the slotted foam organizer can be pigmented with different colors to allow further organization during operating room procedures.

The principles, preferred embodiments and modes of operation of the presently disclosed instrument organizer have been described in the foregoing specification. The presently disclosed instrument organizer, however, is not to be construed as limited to the particular embodiments shown, as these embodiments are regarded as illustrious rather than restrictive. Moreover, variations and changes may be made by those skilled in the art without departing from the spirit and scope of the slotted foam instrument organizer and disclosed herein and recited in the appended claims.

1. An instrument organizer for supporting surgical instruments, comprising:
a polymeric sheet having a predetermined width having a top planar surface and a bottom planar surface;
a protruding polymeric surface position upon said top planar surface wherein said protruding foam surface is attached to said top planar surfaces and said protruding planar surface has an exterior surface and an interior surface wherein said interior surface and said top planar surface form a slotted area that is flexibly hinged to said polymeric sheet.

2. The instrument organizer according to claim 1, wherein said instrument organizer supports ringed handled surgical instruments.

3. The instrument organizer according to claim 1 wherein said bottom planar surface has an adhesive strip.

4. The instrument organizer according to claim 1 wherein said bottom planar surface has a Velcro strip.

5. The instrument organizer according to claim 1 wherein said bottom planar surface has a removable tack adhesive.

6. The instrument organizer according to claim 1 wherein said interior surface and said top planar surface form a slotted area that is flexibly hinged to said foam sheet allowing for the organization of ringed handle surgical instruments.

7. The instrument organizer according to claim 1, wherein said slotted area has a means for organization of ringed handle surgical instruments.

8. The instrument organizer according to claim 1, wherein said instrument organizer is formed from a polymeric material that is lint free.

9. The instrument organizer according to claim 1, wherein said instrument organizer is sterile.

10. The instrument organizer according to claim 1, wherein said instrument organizer is non-sterile.

11. The instrument organizer according to claim 8, wherein said polymeric material is pigmented allowing for further organization based upon color.

12. The instrument organizer according to claim 1, wherein said slotted area allows for organization of non-ringed handle surgical instruments.

13. The instrument organizer according to claim 1, wherein said top planar surface has graphics further allowing for organization of said surgical instruments.

14. The instrument organizer according to claim 1, wherein said polymeric sheet is formed from a material having anti-microbial properties.

15. The instrument organizer according to claim 1, wherein said polymeric sheet is formed from a material that is heat resistant allowing for sterilization of organized surgical instruments.
